(19) United States
(12) Patent Application Publication
(10) Pub. No.: US 2011/0136806 A1
(43) Pub. Date: Jun. 9, 2011

(54) Bicyclic heterocycles, pharmaceutical compositions containing these compounds, the use thereof and processes for the preparation thereof

(75) Inventors: Frank HIMMELSBACH, Mittelbiberach (DE); Birgit JUNG, Laupheim (DE)

(73) Assignee: BOEHRINGER INGELHEIM INTERNATIONAL GMBH, Ingelheim am Rhein (DE)

(21) Appl. No.: 13/027,669

(22) Filed: Feb. 15, 2011

Related U.S. Application Data
(63) Continuation of application No. 11/996,886, filed on Mar. 5, 2008.

(30) Foreign Application Priority Data
Aug. 22, 2005 (EP) 05107679.2

(51) Int. Cl. A61K 31/5377; C07D 401/12; C07D 413/14; C07D 239/94; A61K 31/517; A61P 1/00; A61P 35/00; A61P 11/00

(52) U.S. Cl. 514/234.5; 544/293; 544/119; 514/266.22; 514/266.4; 514/266.2

(57) ABSTRACT

The invention relates to bicyclic heterocycles of general formula (I), in which R^{a}, R^{b}, R^{c}, R^{d}, R^{e} and X are as defined in claim 1, the tautomers, stereoisomers, mixtures and salts thereof, in particular, the physiologically-acceptable salts thereof with inorganic and organic acids with useful pharmacological properties, in particular, an inhibitory effect on signal transduction brought about by tyrosine kinases, the use thereof for the treatment of diseases, in particular of tumour diseases and benign prostatic hyperplasia (BPH), diseases of the lungs and airways and production thereof.

The present invention relates to bicyclic heterocycles of general formula

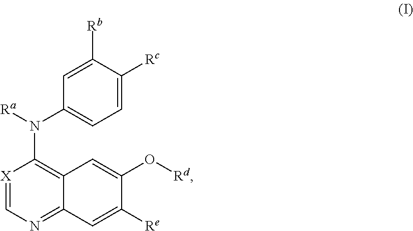

their tautomers, their stereoisomers, their mixtures and their salts, in particular their physiologically acceptable salts with inorganic or organic acids, which have valuable pharmacological properties, in particular an inhibitory action on the signal transduction mediated by tyrosine kinases, their use for the treatment of illnesses, in particular of tumoral diseases and of benign prostatic hyperplasia (BPH), of diseases of the lung and of the airways, and the preparation thereof.

In the above general formula I

R^{a} denotes a hydrogen atom or a C₁₋₃-alkyl group,
R^{b} denotes a hydrogen, fluorine, chlorine, bromine or iodine atom,
a C₁₋₃-alkyl, hydroxy, C₁₋₃-alkoxy, C₂₋₃-alkenyl or C₂₋₃-alkynyl group,
a methyl or methoxy group substituted by 1 to 3 fluorine atoms or
a cyano, nitro or amino group,
R^{c} denotes a hydrogen, fluorine, chlorine or bromine atom, or
a methyl or trifluoromethyl group,
R^{d} denotes a cyclobutyl, cyclopentyl or cyclohexyl group which is substituted in each case by a group R¹—N—R², wherein
R¹ denotes a hydrogen atom or a C₁₋₃-alkyl group and
R² denotes a hydroxy-C₁₋₄-alkyl-carbonyl group,
   an azetidin-3-yl group which is substituted in the 1-position by the group R², where R² is as hereinbefore defined,
   a pyrrolidin-3-yl group which is substituted in the 1-position by the group R², where R² is as hereinbefore defined,
   a piperidin-3-yl group which is substituted in the 1-position by the group R², where R² is as hereinbefore defined, or
   a piperidin-4-yl group which is substituted in the 1-position by the group R², where R² is as hereinbefore defined,
   R^{e} denotes a hydrogen atom or a fluorine, chlorine or bromine atom,
   a hydroxy group,
   a C₁₋₄-alkyloxy group,
   a methoxy group substituted by 1 to 3 fluorine atoms,
   an ethyloxy group substituted by 1 to 5 fluorine atoms,
   a C₂₋₄-alkyloxy group which is substituted by a group R³ or R⁴, where
R³ denotes a 2-oxo-pyrrolidin-1-yl, 2-oxopiperidin-1-yl, 3-oxo-morpholin-4-yl, 2-oxo-imidazolidin-1-yl, 2-oxo-3-C₁₋₃-alkyl-imidazolidin-1-yl, 2-oxo-hexahydro-pyrimidin-1-yl or a 2-oxo-3-C₁₋₃-alkyl-hexahydropyrimidin-1-yl group, and
R⁴ denotes a hydroxy, C₁₋₃-alkyloxy, C₃₋₆-cycloalkyloxy, amino, C₁₋₃-alkylamino, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo-[3.2.1]oct-3-yl, piperazin-1-yl, 4-C₁₋₃-alkyl-piperazin-1-yl, homopiperazin-1-yl or C₁₋₃-alkyl-homopiperazin-1-yl group, or
a formylamino, C₁₋₄-alkylcarbonylamino, C₁₋₃-alkyloxy-C₁₋₃-alkyl-carbonylamino, C₁₋₄-alkyloxycarbonylamino, aminocarbonylamino, C₁₋₃-alkylaminocarbonylamino, di-(C₁₋₃-alkyl)aminocarbonylamino, pyrrolidin-1-ylcarbonylamino, piperidin-1-ylcarbonylamino, piperazin-1-ylcarbonylamino, 4-C₁₋₃-alkyl-piperazin-1-ylcarbonylamino, morpholin-4-ylcarbonylamino or a C₁₋₄-alkylsulphonylamino group,
   a C₃₋₇-cycloalkyloxy or C₃₋₇-cycloalkyl-C₁₋₄-alkyloxy group,
   a tetrahydrofuran-3-yloxy, tetrahydropyran-3-yloxy or tetrahydropyran-4-yloxy group,
   a tetrahydrofuranyl-C₁₋₄-alkyloxy or tetrahydropyranyl-C₁₋₄-alkyloxy group,
   a C₁₋₄-alkoxy group which is substituted by a pyrrolidinyl, piperidinyl or homopiperidinyl group substituted in the 1 position by the group R⁵, wherein
R⁵ denotes a hydrogen atom or a C₁₋₃-alkyl group,
   or a C₁₋₄-alkoxy group which is substituted by a morpholinyl group substituted in the 4 position by the group R⁵, wherein R⁵ is as hereinbefore defined, and
   X denotes a methyne group substituted by a cyano group, or a nitrogen atom, and
   the above-mentioned pyrrolidinyl, piperidinyl, piperazinyl and morpholinyl groups may each be substituted by one or two C₁₋₃-alkyl groups, and
   unless stated otherwise, the above-mentioned alkyl groups may be straight-chain or branched.

Preferred compounds of the above general formula I are those wherein

R^{a} denotes a hydrogen atom,
R^{b} denotes a fluorine, chlorine or bromine atom,
a methyl, trifluoromethyl or ethynyl group,
R^{c} denotes a hydrogen or fluorine atom,
R^{d} denotes a cyclopentyl group which is substituted in the 3-position by a group R¹—N—R², wherein
R¹ denotes a hydrogen atom or a C₁₋₃-alkyl group and
R² denotes a hydroxy-C₁₋₃-alkyl-carbonyl group,
   a cyclohexyl group which is substituted in the 3-position or in the 4-position by a group R¹—N—R², wherein R¹ and R² are as hereinbefore defined,
   a pyrrolidin-3-yl group which is substituted in the 1-position by the group R², wherein R² is as hereinbefore defined,
   a piperidin-3-yl group which is substituted in the 1-position by the group R², wherein R² is as hereinbefore defined,
   a piperidin-4-yl group which is substituted in the 1-position by the group R², wherein R² is as hereinbefore defined,
   R^{e} denotes a hydrogen atom,
   a C₁₋₃-alkyloxy group,
   a methoxy group which is substituted by one to three fluorine atoms,
   an ethyloxy group which is substituted in the 2-position by a group R³ or R⁴, wherein
R³ denotes a 2-oxo-pyrrolidin-1-yl, 2-oxopiperidin-1-yl, 3-oxo-morpholin-4-yl, 2-oxo-imidazolidin-1-yl, 2-oxo-3-methyl-imidazolidin-1-yl, 2-oxo-hexahydro-pyrimidin-1-yl or a 2-oxo-3-methyl-hexahydropyrimidin-1-yl group, and
R⁴ denotes a hydroxy, C₁₋₃-alkyloxy, amino, C₁₋₃-alkylamino, di-(C₁₋₃-alkyl)amino, bis-(2-methoxyethyl)-amino, pyrrolidin-1-yl, piperidin-1-yl, morpholin-4-yl, homomorpholin-4-yl, 2-oxa-5-aza-bicyclo[2.2.1]hept-5-yl, 3-oxa-8-aza-bicyclo-[3.2.1]oct-8-yl, 8-oxa-3-aza-bicyclo[3.2.1]oct-3-yl, piperazin-1-yl or a 4-C₁₋₃-alkyl-piperazin-1-yl group, or
a formylamino, C₁₋₄-alkylcarbonylamino, C₁₋₃-alkyloxy-C₁₋₃-alkyl-carbonylamino, C₁₋₄-alkyloxycarbonylamino, aminocarbonylamino, C₁₋₃-alkylaminocarbonylamino, di-(C₁₋₃-alkyl)aminocarbonylamino, pyrrolidin-1-ylcarbonylamino, piperidin-1-ylcarbonylamino, piperazin-1-ylcarbonylamino, 4-C₁₋₃-alkyl-piperazin-1-ylcarbonylamino-morpholin-4-ylcarbonylamino or a C₁₋₄-alkylsulphonylamino group,
   a propyloxy group which is substituted in the 3-position by a group R³ or R⁴, wherein R³ and R⁴ are as hereinbefore defined, or
   a butyloxy group which is substituted in the 4-position by a group R³ or R⁴, wherein R³ and R⁴ are as hereinbefore defined, and
   X denotes a nitrogen atom,
   while, unless stated otherwise, the above-mentioned alkyl groups may be straight-chain or branched,
   the tautomers, the stereoisomers, the mixtures thereof and the salts thereof.

Particularly preferred compounds of the above general formula I are those wherein

R^{a} denotes a hydrogen atom,
R^{b} denotes a fluorine, chlorine or bromine atom,
a methyl or ethynyl group,
R^{c} denotes a hydrogen or fluorine atom,
R^{d} denotes a cyclohexyl group which is substituted in the 3 position or in the 4-position by a group R¹—N—R², where
R¹ denotes a hydrogen atom, a methyl or ethyl group and
R² denotes a hydroxy-C₁₋₃-alkyl-carbonyl group,
   a pyrrolidin-3-yl group which is substituted in the 1-position by the group R², wherein R² is as hereinbefore defined,
   a piperidin-3-yl group which is substituted in the 1 position by the group R², wherein R² is as hereinbefore defined,
   a piperidin-4-yl group which is substituted in the 1 position by the group R², wherein R² is as hereinbefore defined,
   R^{e} denotes a hydrogen atom,
   a methoxy, difluoromethoxy or ethyloxy group,
   an ethyloxy group which is substituted in the 2 position by a group R³ or R⁴, wherein
R³ denotes a 2-oxo-pyrrolidin-1-yl, 2-oxopiperidin-1-yl, 3-oxo-morpholin-4-yl, 2-oxo-imidazolidin-1-yl, 2-oxo-3-methyl-imidazolidin-1-yl, 2-oxo-hexahydropyri-midin-1-yl or a 2-oxo-3-methyl-hexahydropyrimidin-1-yl group, and
R⁴ denotes a hydroxy, methoxy, ethoxy, amino, dimethylamino, diethylamino, bis-(2-methoxyethyl)-amino, pyrrolidin-1-yl, piperidin-1-yl, morpholin-4-yl, homo-morpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo[3.2.1]oct-3-yl, piperazin-1-yl, 4-methylpiperazin-1-yl or 4-ethylpiperazin-1-yl group, or
an acetylamino, ethylcarbonylamino, propylcarbonylamino, butylcarbonylamino, methoxyacetylamino, butyloxycarbonylamino, ethylaminocarbonylamino, dimethylaminocarbonylamino, pyrrolidin-1-ylcarbonylamino, piperidin-1-ylcarbonylamino, morpholin-4-ylcarbonylamino, methylsulphonylamino, ethylsulphonylamino or butylsulphonylamino group,
   a propyloxy group which is substituted in the 3-position by a group R³ or R⁴, wherein R³ and R⁴ are as hereinbefore defined, or
   a butyloxy group which is substituted in the 4-position by a group R³ or R⁴, wherein R³ and R⁴ are as hereinbefore defined, and
   X denotes a nitrogen atom,
   while, unless stated otherwise, the above-mentioned alkyl groups may be straight-chain or branched,
   the tautomers, the stereoisomers, the mixtures thereof and the salts thereof.

Most particularly preferred compounds of general formula I are those wherein

R^{a} denotes a hydrogen atom,
the phenyl group substituted by R^{b} and R^{c} is a 3-bromophenyl, 3,4-difluorophenyl, 3-chloro-4-fluoro-phenyl or a 3-ethynylphenyl group,
R^{d} denotes a cyclohexyl group which is substituted in the 4 position by a hydroxyacetylamino or N-(hydroxyacetyl)-methylamino group,
a pyrrolidin-3-yl group which is substituted in the 1-position by a hydroxyacetyl group,
a piperidin-3-yl group which is substituted in the 1-position by a hydroxyacetyl group,
a piperidin-4-yl group which is substituted in the 1-position by a hydroxyacetyl group,
R^{e} denotes a hydrogen atom,
a methoxy or ethyloxy group,
an ethyloxy group which is substituted in the 2-position by a group R⁴, wherein
R⁴ denotes a hydroxy, methoxy, ethoxy, amino, dimethylamino, diethylamino, pyrrolidin-1-yl, piperidin-1-yl, morpholin-4-yl, piperazin-1-yl, 4-methylpiperazin-1-yl or 4-ethylpiperazin-1-yl group,
   a propyloxy group which is substituted in the 3-position by a group R⁴, wherein R⁴ is as hereinbefore defined, and
   X denotes a nitrogen atom,
   the tautomers, the stereoisomers, the mixtures thereof and the salts thereof.

Particularly preferred compounds of general formula I are those wherein

R^{a} denotes a hydrogen atom,
the phenyl group substituted by R^{b} and R^{c} is a 3-chloro-4-fluoro-phenyl group or a 3-ethynylphenyl group,
R^{d} denotes a cyclohexyl group which is substituted in the 4 position by a hydroxyacetylamino or N-(hydroxyacetyl)-methylamino group,
a piperidin-4-yl group which is substituted in the 1-position by a hydroxyacetyl group,
R^{e} denotes a hydrogen atom,
a methoxy group, an ethyloxy group or a 2-(methoxy)-ethyloxy group, or
a 2-(morpholin-4-yl)ethyloxy or 3-(morpholin-4-yl)propyloxy group,
and
X denotes a nitrogen atom,
the tautomers, the stereoisomers, the mixtures thereof and the salts thereof.

Of the bicyclic heterocycles of general formula I described above and those substituted-groups which are described in each case as being preferred, particularly preferred, most particularly preferred and especially preferred, special mention should be made of those compounds wherein

(a) R^{d} denotes a cyclohexyl group substituted in the 4-position,
(b) R^{d} denotes a pyrrolidin-3-yl group substituted in the 1-position,
(c) R^{d} denotes a piperidin-3-yl group substituted in the 1-position,
(d) R^{d} denotes a piperidin-4-yl group substituted in the 1-position,
while R^{a}, R^{b}, R^{c}, R^{e} and X in each case are as hereinbefore defined.

Mention may be made, for example, of the following particularly preferred compounds of general formula I:
(1) 4-[(3-chloro-4-fluoro-phenyl)amino]-6-{1-[(hydroxymethyl)carbonyl]-piperidin-4-yl-oxy}-7-methoxy-quinazoline and
(2) 4-[(3-ethynyl-phenyl)amino]-6-{1-[(hydroxymethyl)carbonyl]-piperidin-4-yloxy}-7-methoxy-quinazoline
   and the salts thereof.

The compounds of general formula I may be prepared for example by the following methods:

a) reacting a compound of general formula

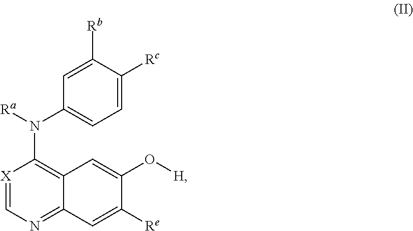

wherein
R^{a}, R^{b}, R^{c}, R^{e} and X are as hereinbefore defined, with a compound of general formula

Z¹—R^{d} (III),

wherein
R^{d} is as hereinbefore defined and Z¹ denotes a leaving group such as a halogen atom, e.g. A chlorine or bromine atom, a sulphonyloxy group such as a methanesulphonyloxy or p-toluenesulphonyloxy group or a hydroxy group.

Using a compound of general formula III in which Z¹ is a hydroxyl group, the reaction is carried out in the presence of a dehydrating agent, preferably in the presence of a phosphine and of an azodicarboxylic acid derivative such as, for example, triphenylphosphine/diethyl azodicarboxylate, expediently in a solvent such as methylene chloride, acetonitrile, tetrahydrofuran, dioxane, toluene or ethylene glycol diethyl ether at temperatures between −50 and 150° C., but preferably at temperatures between −20 and 80° C.

b) For the preparation of compounds of general formula I, in which R^{e} is one of the optionally substituted alkyloxy groups mentioned at the outset:
reacting a compound of general formula

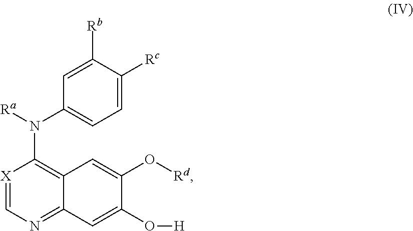

wherein R^{a}, R^{b}, R^{c}, R^{d} and X are as hereinbefore defined, with a compound of general formula

Z²—R^{a} (V),

wherein R^{e′} denotes a C₁₋₄-alkyl group, a methyl group substituted by 1 to 3 fluorine atoms, an ethyl group substituted by 1 to 5 fluorine atoms, a C₂₋₄-alkyl group substituted by a group R³ or R⁴, wherein R³ and R⁴ are as hereinbefore defined, a C₁₋₄-alkyl group which is substituted by a pyrrolidinyl, piperidinyl or homopiperidinyl group substituted in the 1 position by the group R⁵, or a C₁₋₄-alkyl group which is substituted by a morpholinyl group substituted in the 4 position by the group R⁵, wherein R⁵ in each case is as hereinbefore defined, and
Z² denotes a leaving group such as a halogen atom, an alkylsulphonyloxy, arylsulphonyloxy or a hydroxy group.

If the leaving group is a halogen atom such as a chlorine, bromine or iodine atom or an alkylsulphonyloxy or arylsulphonyloxy group such as the methanesulphonyloxy or p-toluenesulphonyloxy group, the reaction is preferably carried out in the presence of an organic or inorganic base such as potassium carbonate, sodium hydride or N-ethyl-diisopropylamine. If the leaving group is a hydroxyl group, then the reaction is carried out in the presence of a dehydrating agent, preferably in the presence of a phosphine and of an azodicarboxylic acid derivative such as, for example, triphenyl-phosphine/diethyl azodicarboxylate.

c) For the preparation of compounds of general formula I, in which R^{e} is one of the alkyloxy groups mentioned at the outset, which is substituted by an optionally substituted amino, alkylamino or dialkylamino group or by an optionally substituted heterocyclic group bonded via an imino nitrogen atom:
reacting a compound of general formula

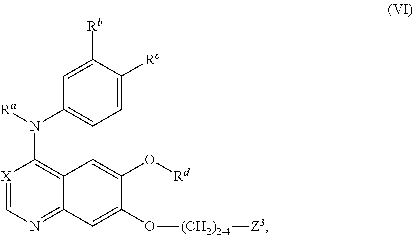

wherein R^{a}, R^{b}, R^{c}, R^{d} and X are as hereinbefore defined and Z³ denotes a leaving group such as a halogen atom, e.g. A chlorine or bromine atom or a sulphonyloxy group such as a methanesulphonyloxy or p-toluenesulphonyloxy group, with
ammonia, a corresponding, optionally substituted alkylamine, dialkylamine or an imino compound or suitable salts or derivatives thereof, such as morpholine, for example.
d) For the preparation of compounds of general formula I wherein R^{e} denotes a hydroxy group:
cleavage of a protective group from a compound of general formula

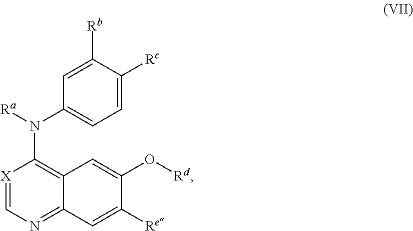

wherein R^{a}, R^{b}, R^{c}, R^{d} and X are as hereinbefore defined and R^{e′} denotes a group which can be converted into a hydroxy group, for example an optionally substituted benzyloxy group, a trimethylsilyloxy, acetyloxy, benzoyloxy, methoxy, ethoxy, tert-butoxy or trityloxy group.

The cleavage of the protective group is carried out, for example, hydrolytically in an aqueous solvent, e.g. In water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. In the presence of iodotrimethylsilane, at temperatures between 0 and 120° C., preferably at temperatures between 10 and 100° C.

The cleavage of a benzyl or methoxybenzyl group is carried out, for example, hydrogenolytically, e.g. With hydrogen in the presence of a catalyst such as pal-ladium/carbon in a suitable solvent such as methanol, ethanol, ethyl acetate or glacial acetic acid optionally with addition of an acid such as hydrochloric acid at temperatures between 0 and 100° C., but preferably at room temperatures between 20 and 60° C., and at a hydrogen pressure of 1 to 7 bar, but preferably of 3 to 5 bar. The cleavage of a 2,4-dimethoxybenzyl group, however, is preferably carried out in trifluoroacetic acid in the presence of anisole.

The cleavage of a tert-butyl or benzyl group is carried out, for example, by treatment with an acid such as trifluoroacetic acid, hydrochloric acid or hydrobromic acid or by treatment with iodotrimethylsilane optionally using a solvent such as methylene chloride, dioxane, methanol or diethyl ether.

e) For the preparation of compounds of general formula I, in which R^{d} contains an amino, alkylamino or imino group substituted by a hydroxyalkyl-carbonyl group:
reacting a compound of general formula

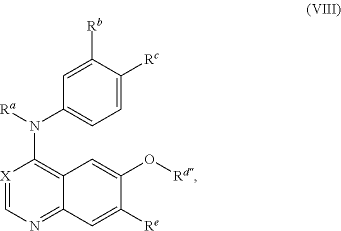

wherein R^{a}, R^{b}, R^{c}, R^{e} and X are as hereinbefore defined and R^{d′} has the meanings given for R^{d} hereinbefore, with the proviso that the hydroxyalkyl-carbonyl group bound to the nitrogen atom of an amino, alkylamino or imino group is replaced by a hydrogen atom, with a hydroxyalkyl-carboxylic acid or a derivative thereof suitable for acylations.

For example the reaction is carried out with a hydroxyalkyl-carboxylic acid in the presence of an activating agent such as N,N-carbonyldiimidazole, N,N′-dicyclohexylcarbodiimide, O-(benzotriazol-1-yl)-N,N,N′N′-tetramethyluronium tetrafluoroborate (TBTU) or O-(7-azabenzotriazol-1-yl)-N,N,N′,N′-tetramethyluronium hexafluorophosphate (HATU), expediently in a solvent such as methylene chloride, dimethylformamide, acetonitrile, tetrahydrofuran, dioxane or ethyleneglycol diethyl ether at temperatures between −50 and 100° C., but preferably at temperatures between −20 and 60° C.

If, according to the invention, a compound of general formula I is obtained which contains an amino, alkylamino or imino group, then this can be converted into a corresponding acyl or sulphonyl compound of general formula I by means of acylation or sulphonylation, where suitable acylating agents are, for example, isocyanates, carbamoyl chlorides, carboxylic acid halides, carboxylic acid anhydrides and carboxylic acids with activating agents such as N,N′-carbonyldiimidazole, N,N′-dicyclohexylcarbodiimide or O-(benzotriazol-1-yl)-N,N,N′N′-tetramethyluronium tetrafluoroborate and suitable sulphonylating agents are sulphonyl halides, and/or

a compound of general formula I, which contains an amino, alkylamino or imino group, then this can be converted into a corresponding alkyl compound of the general formula I by means of alkylation or reductive alkylation and/or
a compound of general formula I, which contains a tert-butyloxycarbonylamino, N-alkyl-N-(tert-butyloxycarbonyl)amino or a N-tert-butyloxycarbonylimino group, then this can be converted into a corresponding amino, alkylamino or imino compound of general formula I by treatment with an acid such as hydrochloric acid or trifluoroacetic acid.

In the reactions described hereinbefore, any reactive groups present such as hydroxy, amino, alkylamino or imino groups may be protected during the reaction by conventional protecting groups which are cleaved again after the reaction.

For example, a protecting group for a hydroxy group may be a trimethylsilyl, tert.butyl-dimethylsilyl, acetyl, trityl, benzyl or tetrahydropyranyl group.

Protecting groups for an amino, alkylamino or imino group may be a formyl, acetyl, trifluoroacetyl, ethoxycarbonyl, tert.butoxycarbonyl, benzyloxycarbonyl, benzyl, methoxybenzyl or 2,4-dimethoxybenzyl group.

Any protecting group used is optionally subsequently cleaved for example by hydrolysis in an aqueous solvent, e.g. In water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. In the presence of iodotrimethylsilane, at temperatures between 0 and 120° C., preferably at temperatures between 10 and 100° C.

However, a benzyl, methoxybenzyl or benzyloxycarbonyl group is cleaved, for example hydrogenolytically, e.g. With hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at ambient temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid or by treating with iodotrimethylsilane optionally using a solvent such as methylene chloride, dioxane, methanol or diethyl ether.

A trifluoroacetyl group is preferably cleaved by treating with an acid such as hydrochloric acid, optionally in the presence of a solvent such as acetic acid at temperatures between 50 and 120° C. or by treating with sodium hydroxide solution, optionally in the presence of a solvent such as tetrahydrofuran or methanol at temperatures between 0 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures obtained may be resolved by chromatography into the cis and trans isomers thereof, the compounds of general formula I obtained which occur as racemates may be separated by methods known per se (cf. Allinger N. L. And Eliel E. L. In “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. By chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. The D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

As already mentioned hereinbefore, the compounds of general formula I according to the invention and the physiologically acceptable salts thereof have valuable pharmacological properties, particularly an inhibiting effect on signal transduction mediated by the Epidermal Growth Factor receptor (EGF-R), whilst this may be achieved for example by inhibiting ligand bonding, receptor dimerisation or tyrosine kinase itself. It is also possible to block the transmission of signals to components located further downstream.

The biological properties of the new compounds were investigated as follows:

The inhibition of human EGF-receptor kinase was determined using the cytoplasmatic tyrosine kinase domain (methionine 664 to alanine 1186, based on the sequence published in Nature 309 (1984), 418). To do this, the protein was expressed in Sf9 insect cells as a GST fusion protein using the Baculovirus expression system.

The enzyme activity was measured in the presence or absence of the test compounds in serial dilutions. The polymer pEY (4:1) produced by SIGMA was used as the substrate. Biotinylated pEY (bio-pEY) was added as the tracer substrate. Every 100 μl of reaction solution contained 10 μl of the inhibitor in 50% DMSO, 20 μl of the substrate solution (200 mM HEPES pH 7.4, 50 mM magnesium acetate, 2.5 mg/ml poly(EY), 5 μg/ml bio-pEY) and 20 μl of enzyme preparation. The enzyme reaction was started by the addition of 50 μl of a 100 μM ATP solution in 10 mM magnesium chloride. The dilution of the enzyme preparation was adjusted so that the incorporation of phosphate into the bio-pEY was linear in terms of time and quantity of enzyme. The enzyme preparation was diluted in 20 mM HEPES pH 7.4, 1 mM EDTA, 130 mM common salt, 0.05% Triton X-100, 1 mM DTT and 10% glycerol.

The enzyme assays were carried out at ambient temperature over a period of 30 minutes and were ended by the addition of 50 μl of a stopping solution (250 mM EDTA in 20 mM HEPES pH 7.4). 100 μl were placed on a streptavidin-coated microtitre plate and incubated for 60 minutes at ambient temperature. Then the plate was washed with 200 μl of a washing solution (50 mM Tris, 0.05% Tween 20). After the addition of 100 μl of a HRPO-labelled anti-PY antibody (PY20H Anti-PTyr:HRP produced by Transduction Laboratories, 250 ng/ml) it was incubated for 60 minutes. Then the microtitre plate was washed three times with 200 μl of washing solution. The samples were then combined with 100 μl of a TMB-peroxidase solution (A:B=1:1, Kirkegaard Perry Laboratories). After 10 minutes the reaction was stopped. The extinction was measured at OD_{450 nm} with an ELISA reader. All data points were measured three times.

The data were matched using an iterative calculation using an analytical programme for sigmoid curves (Graph Pad Prism Version 3.0) with variable Hill pitch. All the iteration data released showed a correlation coefficient of more than 0.9 and the upper and lower values of the curves showed a spread of at least a factor of 5. The concentration of active substance which inhibits the activity of EGF-receptor kinase by 50% (IC₅₀) was derived from the curves. The compounds according to the invention had IC₅₀ values of less than 1000 nM, preferably less than 100 nM.

The compounds of general formula I according to the invention thus inhibit signal transduction by tyrosine kinases, as demonstrated by the example of the human EGF receptor, and are therefore useful for treating pathophysiological processes caused by hyperfunction of tyrosine kinases. These are e.g. Benign or malignant tumours, particularly tumours of epithelial and neuroepithelial origin, metastasisation and the abnormal proliferation of vascular endothelial cells (neoangiogenesis).

The compounds according to the invention are also useful for preventing and treating diseases of the airways and lungs which are accompanied by increased or altered production of mucus caused by stimulation by tyrosine kinases, e.g. In inflammatory diseases of the airways such as chronic bronchitis, chronic obstructive bronchitis, asthma, bronchiectasis, allergic or non-allergic rhinitis or sinusitis, cystic fibrosis, α1-antitrypsin deficiency, or coughs, pulmonary emphysema, pulmonary fibrosis and hyperreactive airways.

The compounds are also suitable for treating diseases of the gastrointestinal tract and bile duct and gall bladder which are associated with disrupted activity of the tyrosine kinases, such as may be found e.g. In chronic inflammatory changes such as cholecystitis, Crohn's disease, ulcerative colitis, and ulcers in the gastrointestinal tract or such as may occur in diseases of the gastrointestinal tract which are associated with increased secretions, such as Ménétrier's disease, secreting adenomas and protein loss syndrome.

In addition, the compounds of general formula I and the physiologically acceptable salts thereof may be used to treat other diseases caused by abnormal function of tyrosine kinases, such as e.g. epidermal hyperproliferation (psoriasis), benign prostatic hyperplasia (BPH), inflammatory processes, diseases of the immune system, hyperproliferation of haematopoietic cells, the treatment of nasal polyps, etc.

By reason of their biological properties the compounds according to the invention may be used on their own or in conjunction with other pharmacologically active compounds, for example in tumour therapy, in monotherapy or in conjunction with other anti-tumour therapeutic agents, for example in combination with topoisomerase inhibitors (e.g. etoposide), mitosis inhibitors (e.g. vinblastine), compounds which interact with nucleic acids (e.g. cis-platin, cyclophosphamide, adriamycin), hormone antagonists (e.g. Tamoxifen), inhibitors of metabolic processes (e.g. 5-FU etc.), cytokines (e.g. Interferons), antibodies, etc. For treating respiratory tract diseases, these compounds may be used on their own or in conjunction with other therapeutic agents for the airways, such as substances with a secretolytic (e.g. Ambroxol, N-acetylcysteine), broncholytic (e.g. Tiotropium or ipratropium or fenoterol, salmeterol, salbutamol) and/or anti-inflammatory activity (e.g. Theophylline or glucocorticoids). For treating diseases in the region of the gastrointestinal tract, these compounds may also be administered on their own or in conjunction with substances having an effect on motility or secretion. These combinations may be administered either simultaneously or sequentially.

For pharmaceutical use the compounds according to the invention are generally used for warm-blooded vertebrates, particularly humans, in doses of 0.01-100 mg/kg of body weight, preferably 0.1-15 mg/kg. For administration they are formulated with one or more conventional inert carriers and/or diluents, e.g. With corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fatty substances such as hard fat or suitable mixtures thereof to produce conventional galenic preparations such as plain or coated tablets, capsules, powders, suspensions, solutions, sprays or suppositories.

The new compounds of formula (I) may be obtained by methods known per se and analogously to the following synthesis examples. The preparation of the starting compounds is described in WO 03/82290 or is carried out using methods known per se.

The Examples that follow are intended to illustrate the present invention in more detail without restricting them:

### PREPARATION OF THE END COMPOUNDS

### Example 1

### 4-[(3-chloro-4-fluoro-phenyl)amino]-6-{1-[(hydroxymethyl)carbonyl]-piperidin-4-yloxy}-7-methoxy-quinazoline

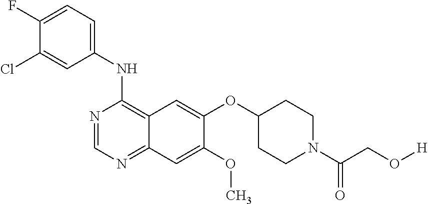

Prepared by reacting 4-[(3-chloro-4-fluoro-phenyl)amino]-6-(piperidin-4-yloxy)-7-methoxy-quinazoline-dihydrochloride with glycolic acid in the presence of N-ethyl-diisopropylamine and O-(benzotriazol-1-yl)-N,N,N′N′-tetramethyluronium tetrafluoroborate (TBTU) at ambient temperature in methylene chloride. R_{f} value: 0.27 (silica gel, methylene chloride/methanol=9:1)

Mass spectrum (ESI⁺): m/z=461, 463 [M+H]⁺

### EXAMPLE 2

### 4-[(3-ethynyl-phenyl)amino]-6-{1-[(hydroxymethyl)carbonyl]-piperidin-4-yloxy}-7-methoxy-quinazoline

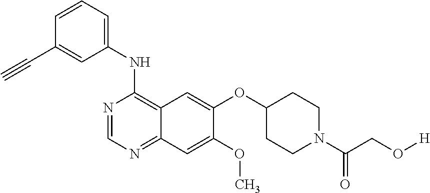

R_{f} value: 0.22 (silica gel, methylene chloride/methanol=9:1)

Mass spectrum (ESI⁺): m/z=434 [M+H]⁺

The following compounds may also be prepared analogously to the above Example and other methods known from the literature:

| Example | |
|---|---|
| No. | Structure |
| (3) | 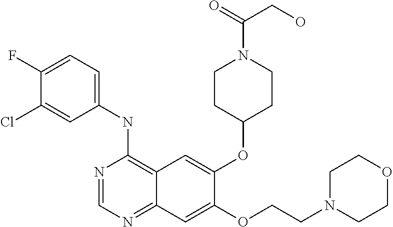 |
| (4) | 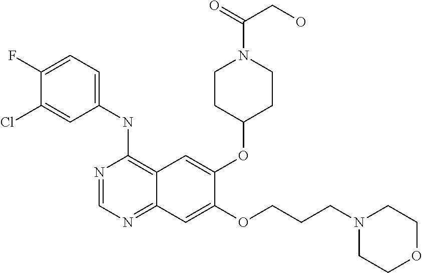 |
| (5) | 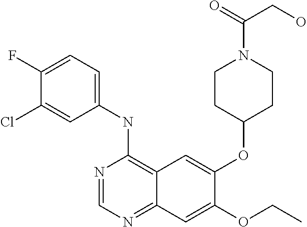 |
| (6) | 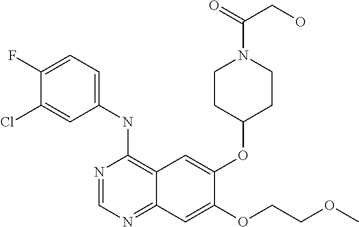 |
| (7) | 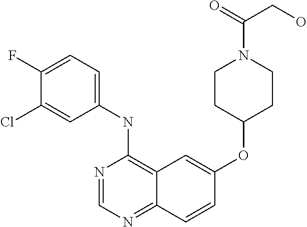 |
| (8) | 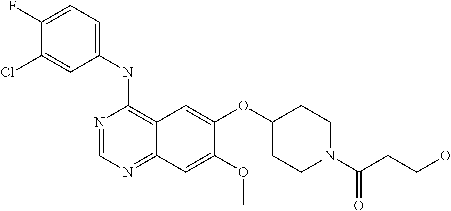 |
| (9) | 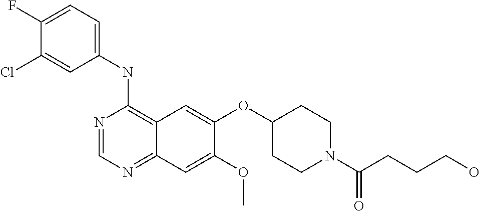 |
| (10) | 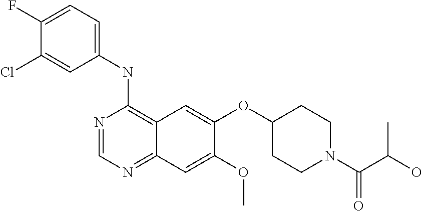 |
| (11) | 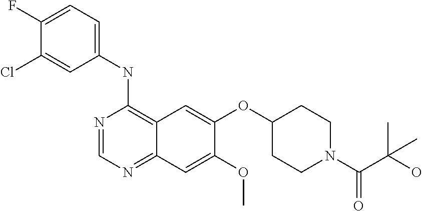 |
| (12) | 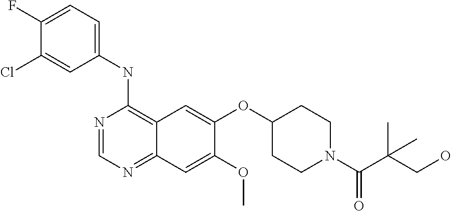 |
| (13) | 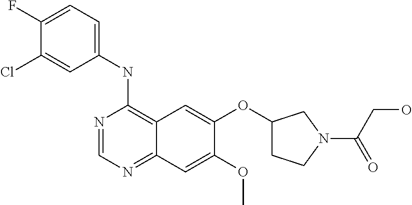 |
| (14) | 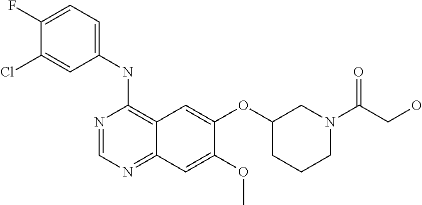 |
| (15) | 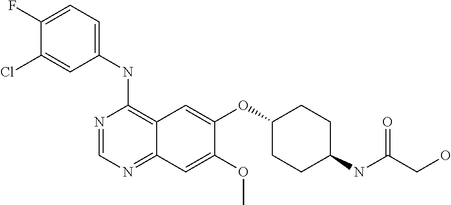 |
| (16) | 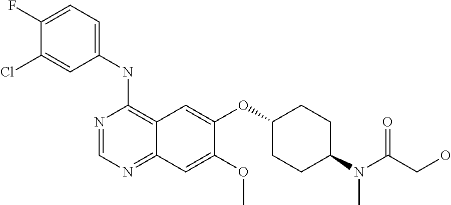 |
| (17) | 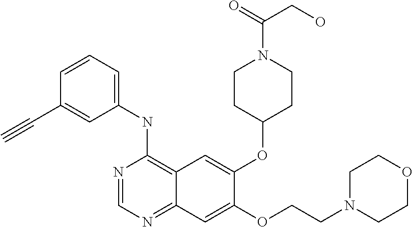 |
| (18) | 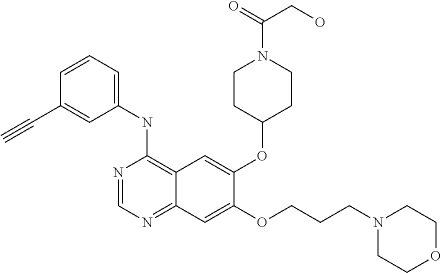 |
| (19) | 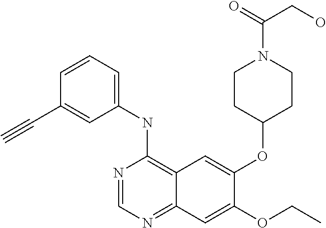 |
| (20) | 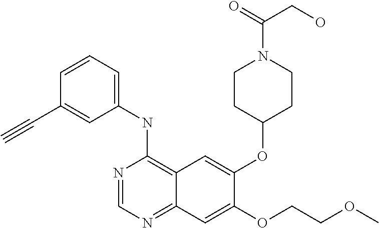 |
| (21) | 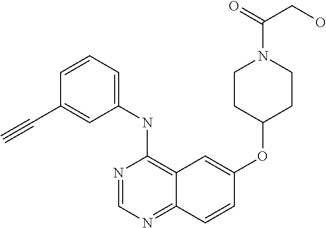 |
| (22) | 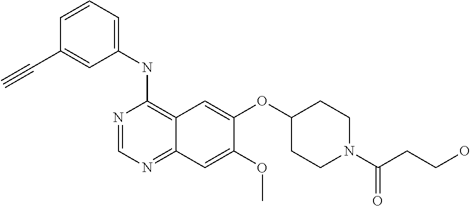 |
| (23) | 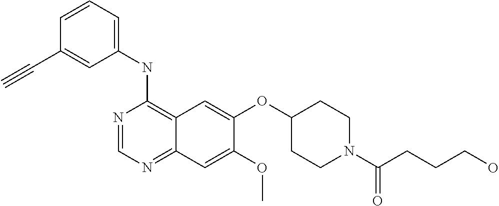 |
| (24) | 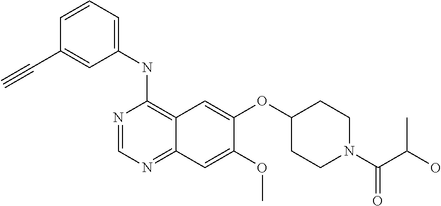 |
| (25) | 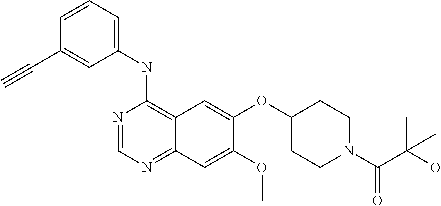 |
| (26) | 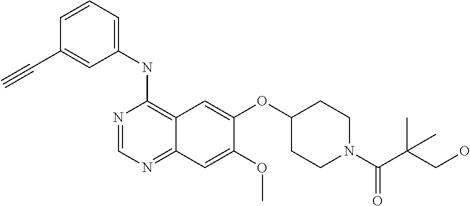 |
| (27) | 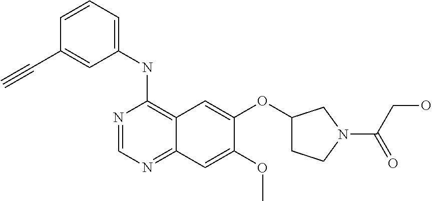 |
| (28) | 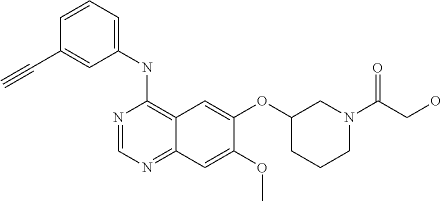 |
| (29) | 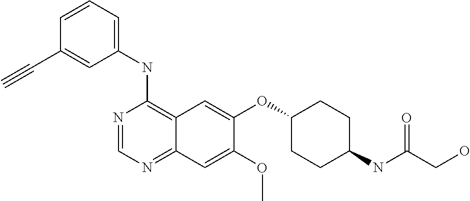 |
| (30) | 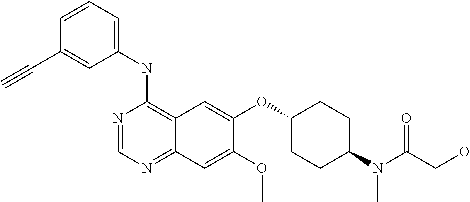 |

The compounds according to the invention may be administered either on their own or in conjunction with other active substances by intravenous, subcutaneous, intramuscular, intraperitoneal or intranasal route, by inhalation or transdermally or orally, whilst aerosol formulations are particularly suitable for inhalation.

The formulation examples that follow illustrate the present invention without restricting its scope.

### A) Coated Tablets Containing 75 mg of Active Substance

1 tablet core contains:

| | |
|---|---|
| active substance | 75.0 mg |
| calcium phosphate | 93.0 mg |
| corn starch | 35.5 mg |
| polyvinylpyrrolidone | 10.0 mg |
| hydroxypropylmethylcellulose | 15.0 mg |
| magnesium stearate | 1.5 mg |
| | 230.0 mg |

### Preparation:

The active substance is mixed with calcium phosphate, corn starch, polyvinylpyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.

| | |
|---|---|
| Weight of core: | 230 mg |
| die: | 9 mm, convex |

The tablet cores thus produced are coated with a film consisting essentially of hydroxypropylmethylcellulose. The finished film-coated tablets are polished with beeswax.
Weight of coated tablet: 245 mg.

### B) Tablets Containing 100 mg of Active Substance

### Composition:

1 tablet contains:

| | |
|---|---|
| active substance | 100.0 mg |
| lactose | 80.0 mg |
| corn starch | 34.0 mg |
| polyvinylpyrrolidone | 4.0 mg |
| magnesium stearate | 2.0 mg |
| | 220.0 mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.

| | |
|---|---|
| Weight of tablet: | 220 mg |
| Diameter: | 10 mm, biplanar, facetted |
| | on both sides and notched on one side. |

### C) Tablets Containing 150 mg of Active Substance

### Composition:

1 tablet contains:

| | |
|---|---|
| active substance | 150.0 mg |
| powdered lactose | 89.0 mg |
| corn starch | 40.0 mg |
| colloidal silica | 10.0 mg |
| polyvinylpyrrolidone | 10.0 mg |
| magnesium stearate | 1.0 mg |
| | 300.0 mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.

| | |
|---|---|
| Weight of tablet: | 300 mg |
| die: | 10 mm, flat |

### D) Hard Gelatine Capsules Containing 150 mg of Active Substance

1 capsule contains:

| | | |
|---|---|---|
| active substance | | 50.0 mg |
| corn starch (dried) | approx. | 80.0 mg |
| lactose (powdered) | approx. | 87.0 mg |
| magnesium stearate | | 3.0 mg |
| | approx. | 420.0 mg |

### Preparation:

The active substance is mixed with the excipients, passed through a screen with a mesh size of 0.75 mm and homogeneously mixed using a suitable apparatus. The finished mixture is packed into size 1 hard gelatine capsules.

| | |
|---|---|
| Capsule filling: | approx. 320 mg |
| Capsule shell: | size 1 hard gelatine capsule. |

### E) Suppositories Containing 150 mg of Active Substance

1 suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| | 2,000.0 mg |

### Preparation:

After the suppository mass has been melted the active substance is homogeneously distributed therein and the melt is poured into chilled moulds.

### F) Suspension Containing 50 mg of Active Substance

100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. Water | ad 100 | ml |

### Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.

5 ml of suspension contain 50 mg of active substance.

### G) Ampoules Containing 10 mg Active Substance

### Composition:

| active substance | 10.0 | mg |
|---|---|---|
| 0.01N hydrochloric acid | q.s. | |
| double-distilled water | ad 2.0 | ml |

### Preparation:

The active substance is dissolved in the requisite amount of 0.01 N HCl, made isotonic with common salt, filtered sterile and transferred into 2 ml ampoules.

### H) Ampoules Containing 50 mg of Active Substance

### Composition:

| active substance | 50.0 | mg |
|---|---|---|
| 0.01N hydrochloric acid | q.s. | |
| double-distilled water | ad 10.0 | ml |

### Preparation:

The active substance is dissolved in the necessary amount of 0.01 N HCl, made isotonic with common salt, filtered sterile and transferred into 10 ml ampoules.

### I) Capsules for Powder Inhalation Containing 5 mg of Active Substance

1 capsule contains:

| | |
|---|---|
| active substance | 5.0 mg |
| lactose for inhalation | 15.0 mg |
| | 20.0 mg |

### Preparation:

The active substance is mixed with lactose for inhalation. The mixture is packed into capsules in a capsule-making machine (weight of the empty capsule approx. 50 mg).

| weight of capsule: | 70.0 mg |
|---|---|
| size of capsule | 3 |

### J) Solution for Inhalation for Hand-Held Nebulisers Containing 2.5 mg Active Substance

1 spray contains:

| active substance | 2.500 | mg |
|---|---|---|
| benzalkonium chloride | 0.001 | mg |
| 1N hydrochloric acid | q.s. | |
| ethanol/water (50/50) | ad 15.000 | mg |

### Preparation:

The active substance and benzalkonium chloride are dissolved in ethanol/water (50/50). The pH of the solution is adjusted with 1N hydrochloric acid. The resulting solution is filtered and transferred into suitable containers for use in hand-held nebulisers (cartridges).

Contents of the container: 4.5 g

1. Bicyclic heterocycles of general formula 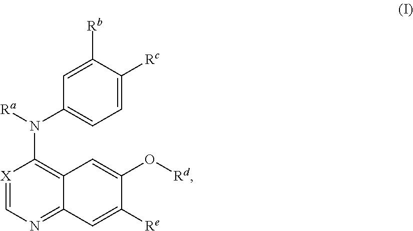
wherein
R^{a} denotes a hydrogen atom or a C₁₋₃-alkyl group,
R^{b} denotes a hydrogen, fluorine, chlorine, bromine or iodine atom,
a C₁₋₃-alkyl, hydroxy, C₁₋₃-alkoxy, C₂₋₃-alkenyl or C₂₋₃-alkynyl group,
a methyl or methoxy group substituted by 1 to 3 fluorine atoms or
a cyano, nitro or amino group,
R^{c} denotes a hydrogen, fluorine, chlorine or bromine atom, or
a methyl or trifluoromethyl group,
R^{d} denotes a cyclobutyl, cyclopentyl or cyclohexyl group which is substituted in each case by a group R¹—N—R², wherein
R¹ denotes a hydrogen atom or a C₁₋₃-alkyl group and
R² denotes a hydroxy-C₁₋₄-alkyl-carbonyl group,
an azetidin-3-yl group which is substituted in the 1-position by the group R², wherein R² is as hereinbefore defined,
a pyrrolidin-3-yl group which is substituted in the 1-position by the group R², wherein R² is as hereinbefore defined,
a piperidin-3-yl group which is substituted in the 1-position by the group R², wherein R² is as hereinbefore defined, or
a piperidin-4-yl group which is substituted in the 1-position by the group R², wherein R² is as hereinbefore defined,
R^{e} denotes a hydrogen atom or a fluorine, chlorine or bromine atom,
a hydroxy group,
a C₁₋₄-alkyloxy group,
a methoxy group substituted by 1 to 3 fluorine atoms,
an ethyloxy group substituted by 1 to 5 fluorine atoms,
a C₂₋₄-alkyloxy group which is substituted by a group R³ or R⁴, where
R³ denotes a 2-oxo-pyrrolidin-1-yl, 2-oxopiperidin-1-yl, 3-oxo-morpholin-4-yl, 2-oxo-imidazolidin-1-yl, 2-oxo-3-C₁₋₃-alkyl-imidazolidin-1-yl, 2-oxo-hexahydro-pyrimidin-1-yl or a 2-oxo-3-C₁₋₃-alkyl-hexahydropyrimidin-1-yl group, and
R⁴ denotes a hydroxy, C₁₋₃-alkyloxy, C₃₋₆-cycloalkyloxy, amino, C₁₋₃-alkylamino, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo-[3.2.1]oct-3-yl, piperazin-1-yl, 4-C₁₋₃-alkyl-piperazin-1-yl, homopiperazin-1-yl or C₁₋₃-alkyl-homopiperazin-1-yl group, or
a formylamino, C₁₋₄-alkylcarbonylamino, C₁₋₃-alkyloxy-C₁₋₃-alkyl-carbonylamino, C₁₋₄-alkyloxycarbonylamino, aminocarbonylamino, C₁₋₃-alkylaminocarbonylamino, di-(C₁₋₃-alkyl)aminocarbonylamino, pyrrolidin-1-ylcarbonylamino, piperidin-1-ylcarbonylamino, piperazin-1-ylcarbonylamino, 4-C₁₋₃-alkyl-piperazin-1-ylcarbonylamino, morpholin-4-ylcarbonylamino or a C₁₋₄-alkylsulphonylamino group,
a C₃₋₇-cycloalkyloxy or C₃₋₇-cycloalkyl-C₁₋₄-alkyloxy group,
a tetrahydrofuran-3-yloxy, tetrahydropyran-3-yloxy or tetrahydropyran-4-yloxy group,
a tetrahydrofuranyl-C₁₋₄-alkyloxy or tetrahydropyranyl-C₁₋₄-alkyloxy group,
a C₁₋₄-alkoxy group which is substituted by a pyrrolidinyl, piperidinyl or homopiperidinyl group substituted in the 1 position by the group R⁵, wherein
R⁵ denotes a hydrogen atom or a C₁₋₃-alkyl group,
or a C₁₋₄-alkoxy group which is substituted by a morpholinyl group substituted in the 4 position by the group R⁵, wherein R⁵ is as hereinbefore defined, and
X denotes a methyne group substituted by a cyano group or a nitrogen atom, and
wherein the above-mentioned pyrrolidinyl, piperidinyl, piperazinyl and morpholinyl groups may each be substituted by one or two C₁₋₃-alkyl groups, and
unless stated otherwise, the above-mentioned alkyl groups may be straight-chain or branched,
the tautomers, the stereoisomers, the mixtures thereof and the salts thereof.

2. Bicyclic heterocycles of general formula I according to claim 1, wherein
R^{a} denotes a hydrogen atom,
R^{b} denotes a fluorine, chlorine or bromine atom,
a methyl, trifluoromethyl or ethynyl group,
R^{c} denotes a hydrogen or fluorine atom,
R^{d} denotes a cyclopentyl group which is substituted in the 3-position by a group R¹—N—R², wherein
R¹ denotes a hydrogen atom or a C₁₋₃-alkyl group and
R² denotes a hydroxy-C₁₋₃-alkyl-carbonyl group,
a cyclohexyl group which is substituted in the 3-position or in the 4-position by a group R¹—N—R², wherein R¹ and R² are as hereinbefore defined,
a pyrrolidin-3-yl group which is substituted in the 1-position by the group R², wherein R² is as hereinbefore defined,
a piperidin-3-yl group which is substituted in the 1-position by the group R², wherein R² is as hereinbefore defined,
a piperidin-4-yl group which is substituted in the 1-position by the group R², wherein R² is as hereinbefore defined,
R^{e} denotes a hydrogen atom,
a C₁₋₃-alkyloxy group,
a methoxy group which is substituted by one to three fluorine atoms,
an ethyloxy group which is substituted in the 2-position by a group R³ or R⁴, wherein
R³ denotes a 2-oxo-pyrrolidin-1-yl, 2-oxopiperidin-1-yl, 3-oxo-morpholin-4-yl, 2-oxo-imidazolidin-1-yl, 2-oxo-3-methyl-imidazolidin-1-yl, 2-oxo-hexahydro-pyrimidin-1-yl or a 2-oxo-3-methyl-hexahydropyrimidin-1-yl group, and
R⁴ denotes a hydroxy, C₁₋₃-alkyloxy, amino, C₁₋₃-alkylamino, di-(C₁₋₃-alkyl)amino, bis-(2-methoxyethyl)-amino, pyrrolidin-1-yl, piperidin-1-yl, morpholin-4-yl, homomorpholin-4-yl, 2-oxa-5-aza-bicyclo[2.2.1]hept-5-yl, 3-oxa-8-aza-bicyclo-[3.2.1]oct-8-yl, 8-oxa-3-aza-bicyclo[3.2.1]oct-3-yl, piperazin-1-yl or a 4-C₁₋₃-alkyl-piperazin-1-yl group, or
a formylamino, C₁₋₄-alkylcarbonylamino, C₁₋₃-alkyloxy-C₁₋₃-alkyl-carbonylamino, C₁₋₄-alkyloxycarbonylamino, aminocarbonylamino, C₁₋₃-alkylaminocarbonylamino, di-(C₁₋₃-alkyl)aminocarbonylamino, pyrrolidin-1-ylcarbonylamino, piperidin-1-ylcarbonylamino, piperazin-1-ylcarbonylamino, 4-C₁₋₃-alkyl-piperazin-1-ylcarbonylamino-morpholin-4-ylcarbonylamino or a C₁₋₄-alkylsulphonylamino group,
a propyloxy group which is substituted in the 3-position by a group R³ or R⁴, wherein R³ and R⁴ are as hereinbefore defined, or
a butyloxy group which is substituted in the 4-position by a group R³ or R⁴, wherein R³ and R⁴ are as hereinbefore defined, and
X denotes a nitrogen atom,
while, unless stated otherwise, the above-mentioned alkyl groups may be straight-chain or branched,
the tautomers, the stereoisomers, the mixtures thereof and the salts thereof.

3. Bicyclic heterocycles of general formula 1 according to claim 1, wherein
R^{a} denotes a hydrogen atom,
R^{b} denotes a fluorine, chlorine or bromine atom,
a methyl or ethynyl group,
R^{c} denotes a hydrogen or fluorine atom,
R^{d} denotes a cyclohexyl group which is substituted in the 3-position or in the 4-position by a group R¹—N—R², where
R¹ denotes a hydrogen atom, a methyl or ethyl group and
R² denotes a hydroxy-C₁₋₃-alkyl-carbonyl group,
a pyrrolidin-3-yl group which is substituted in the 1-position by the group R², wherein R² is as hereinbefore defined,
a piperidin-3-yl group which is substituted in the 1-position by the group R², wherein R² is as hereinbefore defined,
a piperidin-4-yl group which is substituted in the 1-position by the group R², wherein R² is as hereinbefore defined,
R^{e} denotes a hydrogen atom,
a methoxy, difluoromethoxy or ethyloxy group,
an ethyloxy group which is substituted in the 2-position by a group R³ or R⁴, wherein
R³ denotes a 2-oxo-pyrrolidin-1-yl, 2-oxopiperidin-1-yl, 3-oxo-morpholin-4-yl, 2-oxo-imidazolidin-1-yl, 2-oxo-3-methyl-imidazolidin-1-yl, 2-oxo-hexahydropyrimidin-1-yl or a 2-oxo-3-methyl-hexahydropyrimidin-1-yl group, and
R⁴ denotes a hydroxy, methoxy, ethoxy, amino, dimethylamino, diethylamino, bis-(2-methoxyethyl)-amino, pyrrolidin-1-yl, piperidin-1-yl, morpholin-4-yl, homo-morpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo[3.2.1]oct-3-yl, piperazin-1-yl, 4-methylpiperazin-1-yl or 4-ethylpiperazin-1-yl group, or
an acetylamino, ethylcarbonylamino, propylcarbonylamino, butylcarbonylamino, methoxyacetylamino, butyloxycarbonylamino, ethylaminocarbonylamino, dimethylaminocarbonylamino, pyrrolidin-1-ylcarbonylamino, piperidin-1-ylcarbonylamino, morpholin-4-ylcarbonylamino, methylsulphonylamino, ethylsulphonylamino or butylsulphonylamino group,
a propyloxy group which is substituted in the 3-position by a group R³ or R⁴, wherein R³ and R⁴ are as hereinbefore defined, or
a butyloxy group which is substituted in the 4-position by a group R³ or R⁴, wherein R³ and R⁴ are as hereinbefore defined, and
X denotes a nitrogen atom,
while, unless stated otherwise, the above-mentioned alkyl groups may be straight-chain or branched,
the tautomers, the stereoisomers, the mixtures thereof and the salts thereof.

4. Bicyclic heterocycles of general formula I according to claim 1, wherein
R^{a} denotes a hydrogen atom,
the phenyl group substituted by R^{b} and R^{c} is a 3-bromophenyl, 3,4-difluorophenyl, 3-chloro-4-fluoro-phenyl or a 3-ethynylphenyl group,
R^{d} denotes a cyclohexyl group which is substituted in the 4-position by a hydroxyacetylamino or N-(hydroxyacetyl)-methylamino group,
a pyrrolidin-3-yl group which is substituted in the 1-position by a hydroxyacetyl group,
a piperidin-3-yl group which is substituted in the 1-position by a hydroxyacetyl group,
a piperidin-4-yl group which is substituted in the 1-position by a hydroxyacetyl group,
R^{e} denotes a hydrogen atom,
a methoxy or ethyloxy group,
an ethyloxy group which is substituted in the 2-position by a group R⁴, wherein
R⁴ denotes a hydroxy, methoxy, ethoxy, amino, dimethylamino, diethylamino, pyrrolidin-1-yl, piperidin-1-yl, morpholin-4-yl, piperazin-1-yl, 4-methylpiperazin-1-yl or 4-ethylpiperazin-1-yl group,
a propyloxy group which is substituted in the 3-position by a group R⁴, wherein R⁴ is as hereinbefore defined, and
X denotes a nitrogen atom,
the tautomers, the stereoisomers, the mixtures thereof and the salts thereof.

5. Bicyclic heterocycles of general formula I according to claim 1, wherein
R^{a} denotes a hydrogen atom,
the phenyl group substituted by R^{b} and R^{c} is a 3-chloro-4-fluoro-phenyl group or a 3-ethynylphenyl group,
R^{d} denotes a cyclohexyl group which is substituted in the 4-position by a hydroxyacetylamino or N-(hydroxyacetyl)-methylamino group,
a piperidin-4-yl group which is substituted in the 1-position by a hydroxyacetyl group,
R^{e} denotes a hydrogen atom,
a methoxy group, an ethyloxy group or a 2-(methoxy)-ethyloxy group, or
a 2-(morpholin-4-yl)ethyloxy or 3-(morpholin-4-yl)propyloxy group,
and
X denotes a nitrogen atom,
the tautomers, the stereoisomers, the mixtures thereof and the salts thereof.

6. The following compounds of general formula I according to claim 1:
(a) 4-[(3-chloro-4-fluoro-phenyl)amino]-6-{1-[(hydroxymethyl)carbonyl]-piperidin-4-yl-oxy}-7-methoxy-quinazoline and
(b) 4-[(3-ethynyl-phenyl)amino]-6-{1-[(hydroxymethyl)carbonyl]-piperidin-4-yloxy}-7-methoxy-quinazoline
and the salts thereof.

7. A physiologically acceptable salt of a compound of claim 1 with inorganic or organic acids.

8. A pharmaceutical composition containing a compound according to claim 1 optionally together with one or more inert carriers and/or diluents.

9. A method for the treatment of benign or malignant tumours, diseases of the airways and lungs or diseases of the gastro-intestinal tract, bile duct and gall bladder comprising administering to a patient in need thereof a therapeutically effective amount of a compound according to claim 1 or a physiologically acceptable salt thereof.

10. A process for preparing a pharmaceutical composition according to claim 8, comprising incorporation of one or more inert carriers and/or diluents by a non-chemical method.

11. A process for preparing a compound of general formula I according to claim 1 comprising
a) reaction of a compound of general formula 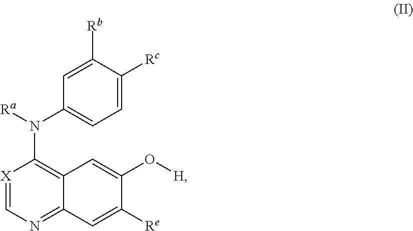
wherein
R^{a}, R^{b}, R^{c}, R^{e} and X are defined as in claim 1 with a compound of general formula
Z¹—R^{d} ,(III)
wherein
R^{d} is defined as in claim 1 and Z¹ denotes a leaving group, or
b) in order to prepare compounds of general formula I wherein R^{e} denotes one of the optionally substituted alkyloxy groups mentioned in claim 1,
a compound of general formula 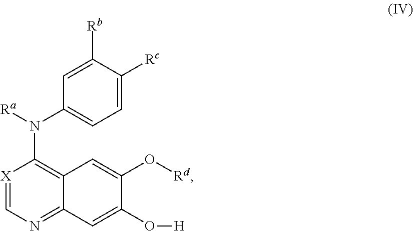
wherein R^{a}, R^{b}, R^{c}, R^{d} and X are defined as in claim 1, is reacted with a compound of general formula
Z²—R^{e′} ,(V)
wherein R^{e′} denotes a C₁₋₄-alkyl group, a methyl group substituted by 1 to 3 fluorine atoms, an ethyl group substituted by 1 to 5 fluorine atoms, a C₂₋₄-alkyl group substituted by a group R³ or R⁴, wherein R³ and R⁴ are defined as in claim 1, a C₁₋₄-alkyl group which is substituted by a pyrrolidinyl, piperidinyl or homopiperidinyl group substituted in the 1 position by the group R⁵, or a C₁₋₄-alkyl group which is substituted by a morpholinyl group substituted in the 4 position by the group R⁵, wherein R⁵ in each case is defined as in claim 1, and Z² denotes a leaving group, or
c) in order to prepare compounds of general formula I wherein R^{e} denotes one of the alkyloxy groups mentioned in claim 1 which is substituted by an optionally substituted amino, alkylamino or dialkylamino group or by an optionally substituted heterocyclic group bound via an imino nitrogen,
a compound of general formula 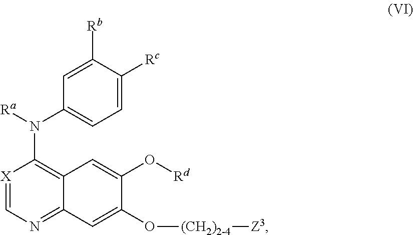
wherein R^{a}, R^{b}, R^{c}, R^{d} and X are defined as in claim 1 and Z³ denotes a leaving group, is reacted with ammonia, a corresponding, optionally substituted alkylamine, dialkylamine or an imino compound or suitable salts or derivatives thereof, or
d) in order to prepare compounds of general formula I wherein R^{e} denotes a hydroxy group,
a protective group is cleaved from a compound of general formula 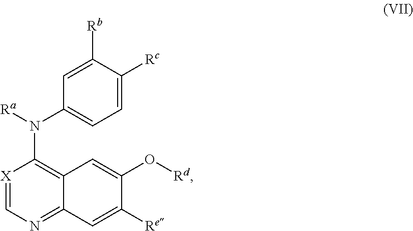
wherein R^{a}, R^{b}, R^{c}, R^{d} and X are defined as in claim 1 and R^{e′} denotes a group which can be converted into a hydroxy group, or
e) in order to prepare compounds of general formula I wherein R^{d} contains an amino, alkylamino or imino group substituted by a hydroxyalkyl-carbonyl group,
a compound of general formula 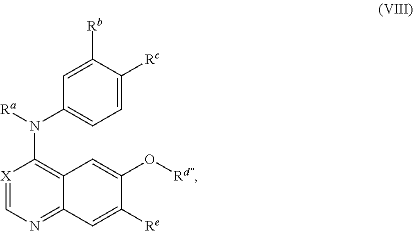
wherein R^{a}, R^{b}, R^{c}, R^{e} and X are defined as in claim 1 and R^{d′} has the meanings given for R^{d} in claim 1, with the proviso that the hydroxyalkyl-carbonyl group bound to the nitrogen atom of an amino, alkylamino or imino group is replaced by a hydrogen atom, is reacted with a hydroxyalkyl-carboxylic acid or a derivative thereof suitable for acylations, and
if desired a compound of general formula I thus obtained which contains an amino, alkylamino or imino group, is converted by acylation or sulphonylation into a corresponding acyl or sulphonyl compound of general formula I and/or
a compound of general formula I thus obtained which contains an amino, alkylamino or imino group is converted by alkylation or reductive alkylation into a corresponding alkyl compound of general formula I and/or
a compound of general formula I thus obtained which contains a tert.-butyloxycarbonylamino, N-alkyl-N-(tert.-butyloxycarbonyl)amino or an N-tert.-butyloxycarbonylimino group is converted by treatment with an acid into a corresponding amino, alkylamino or imino compound of general formula I, and/or
if necessary any protective group used during the reactions described hereinbefore is cleaved again and/or
if desired a compound of general formula I thus obtained is resolved into its stereoisomers and/or
a compound of general formula I thus obtained is converted into its salts, particularly for pharmaceutical use into the physiologically acceptable salts thereof.
